(19) United States
(12) Patent Application Publication
(10) Pub. No.: US 2026/0053777 A1
(43) Pub. Date: Feb. 26, 2026

(54) USE OF NRF2 ACTIVATORS FOR THE TREATMENT OF CEREBRAL SMALL VESSEL DISEASE

(71) Applicants: INSTITUT NATIONAL DE LA SANTÉ ET DE LA RECHERCHE MÉDICALE, Paris (FR); UNIVERSITÉ DE BORDEAUX, Bordeaux (FR); CHU DE BORDEAUX, Talence (FR)

(72) Inventors: Thierry COUFFINHAL, Pessac (FR); Aniket MISHRA, Bordeaux Cedex (FR); Cecile DUPLAA, Pessac Cedex (FR); Claire PEGHAIRE, Pessac Cedex (FR); Stéphanie DEBETTE, Bordeaux (FR)

(21) Appl. No.: 19/105,461

(22) PCT Filed: Sep. 1, 2023
(86) PCT No.: PCT/EP2023/074089

§ 371 Date: Feb. 21, 2025

(30) Foreign Application Priority Data
Sep. 2, 2022 (EP) 22306304.1

(51) Int. Cl. A61K 31/404; A61K 31/122; A61K 31/225; A61K 31/4015; A61P 9/00; A61P 25/00

(52) U.S. Cl. CPC A61K 31/404; A61K 31/122; A61K 31/225; A61K 31/4015; A61P 9/00; A61P 25/00

(57) ABSTRACT

Cerebral small vessel disease (SVD) is a leading cause of stroke and a major contributor to cognitive decline and dementia in the population. Evidences indicate that blood brain barrier dysfunction may play a significant role in VD pathogenesis. Recently, an inverse association of TRIM47 expression in brain and vascular tissues with extensive-SVD severity was reported in a human genome wide association study combined with summary-based Mendelian randomization studies and profiling of human loss-of-function allele carriers. Now, the inventors demonstrate TRIM47 is a key regulator of actin cytoskeleton organization through KEAP1/NRF2 signalling pathway and might be protective from oxidative stress in brain EC. In particular, the in vitro TRIM47 knockdown decreases directed EC migration and delays EC adhesion process with loss of actin cortical reorganization and focal adhesion contacts. Furthermore, RNA sequencing and BioID results indicate that TRIM47 knockdown in brain EC, represses the expression of genes associated with cytoskeleton and NRF2 antioxidant pathway through a potential interaction with KEAP1. Accordingly, the present invention relates to the use of Nrf2 activators for the treatment of SVD.

### FIELD OF THE INVENTION

The present invention is in the field of medicine, in particular microvascular biology.

### BACKGROUND OF THE INVENTION

Cerebral small vessel disease encompasses a group of pathological processes affecting small arteries, arterioles, capillaries and small veins in the brain. It is one of the main causes of stroke, representing a quarter to a third of stroke cases, importantly both ischaemic and haemorrhagic stroke (Greenberg S M. Small vessels, big problems. N Engl J Med 2006; 354(14):1451-1453; Wardlaw J M, Smith C, Dichgans M. Small vessel disease: Mechanisms and clinical implications. Lancet Neurol. 2019; 18(7):684-696). Cerebral small vessel disease features on brain MRI have been associated with an increased risk of dementia, including of the Alzheimer type, and with accelerated cognitive decline (Wardlaw J M, Smith C, Dichgans M. Small vessel disease: Mechanisms and clinical implications. Lancet Neurol. 2019; 18(7):684-696). In fact, the vast majority of cognitive impairment and dementia cases in the community are now largely recognized as due to a mix of neurodegenerative processes and vascular brain injury, of which cerebral small vessel disease is by far the most important substrate (Viswanathan A, Rocca W A, Tzourio C. Vascular risk factors and dementia: How to move forward?Neurology. 2009; 72(4):368-374; Alber J, Alladi S, Bae H J, et al., White matter hyperintensities in vascular contributions to cognitive impairment and dementia (VCID): Knowledge gaps and opportunities. Alzheimers Dement (N Y). 2019; 5:107-117). No specific mechanistic treatments are available for cerebral small vessel disease to date.

A recent gene-mapping study of extremes of cerebral small vessel disease reveals a putative role of TRIM47 in the pathophysiology of said disease (Mishra A, Duplaà C, Vojinovic D, Suzuki H, Sargurupremraj M, Zilhão N R, Li S, Bartz T M, Jian X, Zhao W, Hofer E, Wittfeld K, Harris S E, van der Auwera-Palitschka S, Luciano M, Bis J C, Adams H H H, Satizabal C L, Gottesman R F, Gampawar P G, Bülow R, Weiss S, Yu M, Bastin M E, Lopez O L, Vernooj M W, Beiser A S, Völker U, Kacprowski T, Soumare A, Smith J A, Knopman D S, Morris Z, Zhu Y, Rotter J I, Dufouil C, Valdis Herndndez M, Muñoz Maniega S, Lathrop M, Boerwinkle E, Schmidt R, Ihara M, Mazoyer B, Yang Q, Joutel A, Tournier-Lasserve E, Launer L J, Deary I J, Mosley T H, Amouyel P, DeCarli C S, Psaty B M, Tzourio C, Kardia S L R, Grabe H J, Teumer A, van Duijn C M, Schmidt H, Wardlaw J M, Ikram M A, Fornage M, Gudnason V, Seshadri S, Matthews P M, Longstreth W T, Couffinhal T, Debette S. Gene-mapping study of extremes of cerebral small vessel disease reveals TRIM47 as a strong candidate. Brain. 2022 Jun. 30; 145(6):1992-2007. doi: 10.1093/brain/awab432. PMID: 35511193; PMCID: PMC9255380).

The transcription factor NF-E2 p45-related factor 2 (NRF2; encoded by NFE2L2) and its principal negative regulator, the E3 ligase adaptor Kelch-like ECH-associated protein 1 (KEAP1), are critical in the maintenance of redox, metabolic and protein homeostasis, as well as the regulation of inflammation (Cuadrado, A., Rojo, A. I., Wells, G. et al. Therapeutic targeting of the NRF2 and KEAP1 partnership in chronic diseases. Nat Rev Drug Discov 18, 295-317 (2019)). Thus, NRF2 activation provides cytoprotection against numerous pathologies including chronic diseases of the lung and liver; autoimmune, neurodegenerative and metabolic disorders; and cancer initiation. However, the interest of NRF2 activator in the treatment of cerebral small vessel disease has never been investigated.

### SUMMARY OF THE INVENTION

The present invention is defined by the claims. In particular, the present invention relates to the use of Nrf2 activators for the treatment of cerebral small vessel disease.

### DETAILED DESCRIPTION OF THE INVENTION

The present invention relates to a method of treating a cerebral small vessel disease in a patient in need thereof comprising administering to the patient a therapeutically effective amount of a NRF2 activator.

As used herein, the term “cerebral small vessel disease” or “CSVD” has its general meaning in the art and refers to a group of several diseases affecting the small arteries, arterioles, venules, and capillaries of the brain, and refers to several pathological processes and etiologies (Li Q, Yang Y, Reis C, Tao T, Li W, Li X, Zhang J H. Cerebral Small Vessel Disease. Cell Transplant. 2018 December; 27(12):1711-1722. doi: 10.1177/0963689718795148). Neuroimaging features of CSVD include recent small subcortical infarcts, lacunes, white matter hyperintensities, perivascular spaces, microbleeds, and brain atrophy. The main clinical manifestations of CSVD include stroke, cognitive decline, dementia, psychiatric disorders, abnormal gait, and urinary incontinence.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is particularly suitable for the treatment of SVD-related vascular cognitive impairment and dementia (VCID). VCID encompasses all types of cerebrovascular cardiovascular disease-related cognitive decline.

As used herein, the terms “Nuclear factor (erythroid-derived 2)-like 2 activator” or “Nrf2 activator” relates to chemical compounds or elements that increase the activity of Nrf2. Nrf2 activators are typically classified based on their chemical structures: Diphenols, Michael reaction acceptors, isothiocyanates, thiocarbamates, trivalent arsenicals, 1,2-dithiole-3-thiones, hydroperoxides, vicinal dimercaptans, heavy metals, and polyenes. nNrf2 activators are well known in the art (Cuadrado, A., Rojo, A. I., Wells, G. et al. Therapeutic targeting of the NRF2 and KEAP1 partnership in chronic diseases. Nat Rev Drug Discov 18, 295-317 (2019)).

In some embodiments, the Nrf2 activator of the present invention is selected from the group consisting of Chalcone derivatives as disclosed in J. Med. Chem., 2011, 54 (12), pp 4147-4159, such as 2-trifluoromethyl-2′-methoxychalcone, auranofin, ebselen, 1,2-naphthoquinone, cynnamic aldehyde, caffeic acid and its esters, curcumin, reservatrol, artesunate, tert-butylhydroquinone, and -quinone, (tBHQ, tBQ), vitamins K1, K2 and K3, preferably menadione, fumaric acid esters, i.e. fumaric acid mono- and/or diester which is preferably selected from the group of monoalkyl hydrogen fumarate and dialkyl fumarate, such as monomethyl hydrogen fumarate, diroximel fumarate, dimethyl fumarate, monoethyl hydrogen fumarate, and diethyl fumarate, 2-cyclopentenones, ethacrynic acid and its alkyl esters, bardoxolone methyl (methyl 2-cyano-3,12-dioxooleana-1,9(11)dien-28-oate) (CDDO-Me, RTA 402), ethyl 2-cyano-3,12-dioxooleana-1,9(11)dien-28-oate, 2-cyano-3,12-dioxooleana-1,9(11)dien-28-oic acid (CDDO), 1 [2-Cyano-3,12-dioxooleana-1,9(11)-dien-28-oyl]imidazole (CDDO-Im), (2-cyano-N-methyl-3,12-dioxooleana-1,9(11)-dien-28 amide (CDDO-methyl amide, CDDO-MA), isothiocyanate such as sulforaphane, 1,2-dithiole-3-thione such as oltipraz, 3,5-di-tert-butyl-4-hydroxytoluene, 3-hydroxycoumarin, 4-hydroxynonenal, 4-oxononenal, malondialdehyde, (E)-2-hexenal, capsaicin, allicin, allylisothiocyanate, 6-methylthiohexyl isothiocyanate, 7-methylthioheptyl isothiocyanate, sulforaphane, 8-methylthiooctyl isothiocyanate, corticosteroids, such as dexamethasone, 8-iso prostaglandin A2, alkyl pyruvate, such as methyl and ethyl pyruvate, diethyl or dimethyl oxaloproprionate, 2-acetamidoacrylate, methyl or ethyl-2-acetamidoacrylate, hypoestoxide, parthenolide, eriodictyol, 4-Hydroxy-2-nonenal, 4-oxo-2nonenal, geranial, zerumbone, aurone, isoliquiritigenin, xanthohumol, [10]-Shogaol, eugenol, 1′-acetoxychavicol acetate, allyl isothiocyanate, benzyl isothiocyanate, phenethyl isothiocyanate, 4-(Methylthio)-3-butenyl isothiocyanate and 6-Methylsulfinylhexyl isothiocyanate, ferulic acid and its esters, such as ferulic acid ethyl ester, and ferulic acid methyl ester, sofalcone, 4-methyl daphnetin, imperatorin, auraptene, poncimarin, bis[2-hydroxybenzylidene]acetones, alicylcurcuminoid, 4-bromo flavone, 3-naphthoflavone, sappanone A, aurones and its corresponding indole derivatives such as benzylidene-indolin-2-ones, perillaldehyde, quercetin, fisetin, koparin, genistein, tanshinone HA, BHA, BHT, PMX-290, AL-1, avicin D, gedunin, fisetin, andrographolide, tricyclic bis(cyano enone) TBE-31 [(±)-(4bS,8aR,10aS)-10a-ethynyl-4-b,8,8-trimethyl-3,7-dioxo-3,4-b,7,8,8a,9,10,10a-octahydrophenanthrene-2,6-dicarbonitrile], MCE-1, MCE5, TP-225, ADT as referred to in in Medicinal Research Reviews, 32, No. 4, 687-726, 2012, and the respective quinone or hydroquinone forms of the aforementioned quinone and hydroquinone derivatives and stereoisomers, tautomers or pharmacologically active derivatives of the aforementioned agents.

In some embodiments, the Nrf2 activator of the present invention is selected from the group consisting of fumaric acid derivatives (Joshi and Strebel, WO 2002/055063, US 2006/0205659, and U.S. Pat. No. 7,157,423 (amide compounds and protein-fumarate conjugates); Joshi et al., WO 2002/055066 and Joshi and Strebel, U.S. Pat. No. 6,355,676 (mono and dialkyl esters); Joshi and Strebel, WO 2003/087174 (carbocyclic and oxacarbocylic compounds); Joshi et al., WO 2006/122652 (thiosuccinates); Joshi et al., US 2008/0233185 (dialkyl and diaryl esters) and salts (Nilsson et al., US 2008/0004344) Controlled release pharmaceutical compositions comprising fumaric acid esters are also disclosed by Nilsson and Willer, WO 2007/042034. Prodrugs are described by Nielsen and Bundgaard, J Pharm Sci 1988, 77(4), 285-298 and in WO2010/022177.

Additional examples of Nrf2 activators can be found in US2011/0250300, US 2004/0002463, US 20130172391, US20140275205, WO2014100728 the disclosures of each of which are hereby incorporated by reference herein.

In some embodiments, the Nrf2 activator is the diroximel fumarate (trade name, Vumerity).

In some embodiments, the Nrf2 activator is the dimethyl fumarate (DMF; trade names, Fumaderm or Tecfidera).

As used herein, the term “therapeutically effective amount” a sufficient amount of the Nrf2 activator of the present invention for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the Nrf2 activator of the present inventions; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the Nrf2 activator of the present invention for the symptomatic adjustment of the dosage to the subject to be treated. A medicament typically contains from about 0.01 mg to about 500 mg of the Nrf2 activator of the present invention, typically from 1 mg to about 100 mg of the Nrf2 activator of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

Typically the Nrf2 activator of the present invention is combined with pharmaceutically acceptable excipients, and optionally sustained-release matrices, such as biodegradable polymers, to form pharmaceutical compositions. The term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the Nrf2 activator of the present inventions of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. TRIM47 regulates NRF2 target genes. HO1 (heme oxygenase 1) and NQO1 (NAPDH quinone dehydrogenase 1). * unpaired T test.

FIG. 2. TRIM47 partly mediates H2O2 induction of NRF2-dependent genes. HO1 (heme oxygenase 1) and NQO1 (NAPDH quinone dehydrogenase 1). H2O2: inducer of oxidative stress. *oneway-ANOVA plus multiple comparisons; #unpaired T test.

FIG. 3. Activation of NRF2 at the protein level partly rescues the effect of TRIM47 knockdown. tert-butylhydroquinone=tBHQ: activator of NRF2 by stabilizing NRF2 protein. * oneway-ANOVA plus multiple comparisons.

FIG. 4. TRIM47 is essential to brain physiology by promoting the NRF2 antioxidant protective system. A. TRIM47 displays antioxidant properties. B. RNA-sequencing was performed on HBMEC treated with control or TRIM47 siRNA. C. Assessment of blood brain barrier (BBB) permeability was done on Trim47 +/+ and Trim47 −/− adult mice. Mann-Whitney test. D. Representative image by immunofluorescence of GFAP expression (activated astrocytes) in brain sections from Trim47 +/+ and Trim47 −/− adult mice. E. Water maze behavioural test performed on adult mice with/without a diet with tBHQ. Two-way ANOVA with repeated measures.

### Example 1

Introduction. Cerebral small vessel disease (SVD) is a leading cause of stroke and a major contributor to cognitive decline and dementia in the population. Evidences indicate that blood brain barrier dysfunction may play a significant role in VD pathogenesis. Recently, an inverse association of TRIM47 expression in brain and vascular tissues with extensive-SVD severity was reported in a human genome wide association study combined with summary-based Mendelian randomization studies and profiling of human loss-of-function allele carriers.

Aims. Our goal is to understand the role of TRIM47 in cerebral endothelial cells (EC).

Methods. TRIM47 interacting effectors were searched by proximity labeling assay (BioID) in human brain EC. TRIM47 knockdown in EC allowed to explore TRIM47 role in functional assays, and to identify transcriptional response by RNA-sequencing.

Results. In vitro, TRIM47 knockdown decreases directed EC migration and delays EC adhesion process with loss of actin cortical reorganization and focal adhesion contacts. Put together, RNA sequencing and BioID results indicate that TRIM47 knockdown in brain EC, represses the expression of genes associated with cytoskeleton and NRF2 antioxidant pathway through a potential interaction with KEAP1, an important player in focal adhesion and cytoskeleton organization.

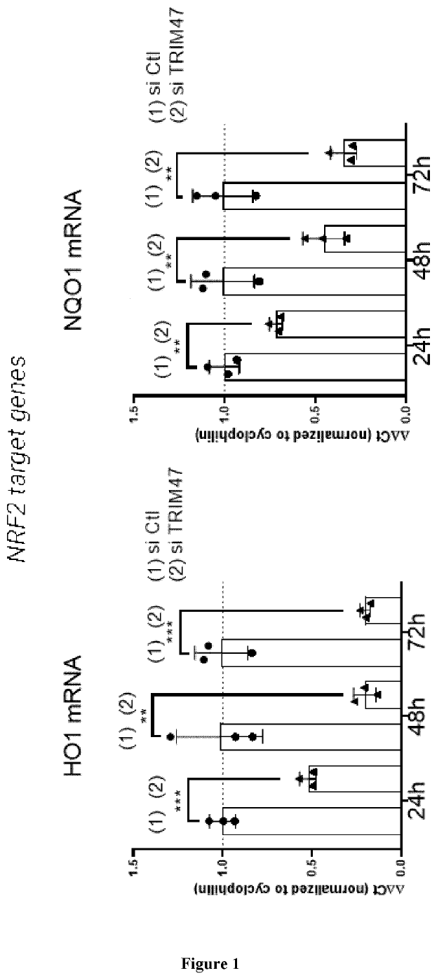

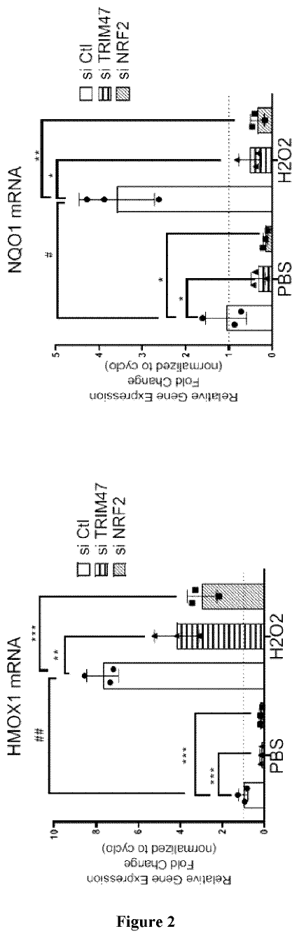

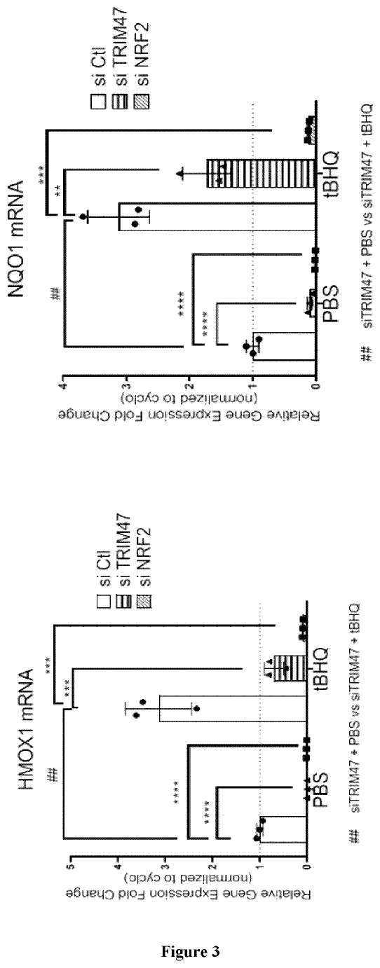

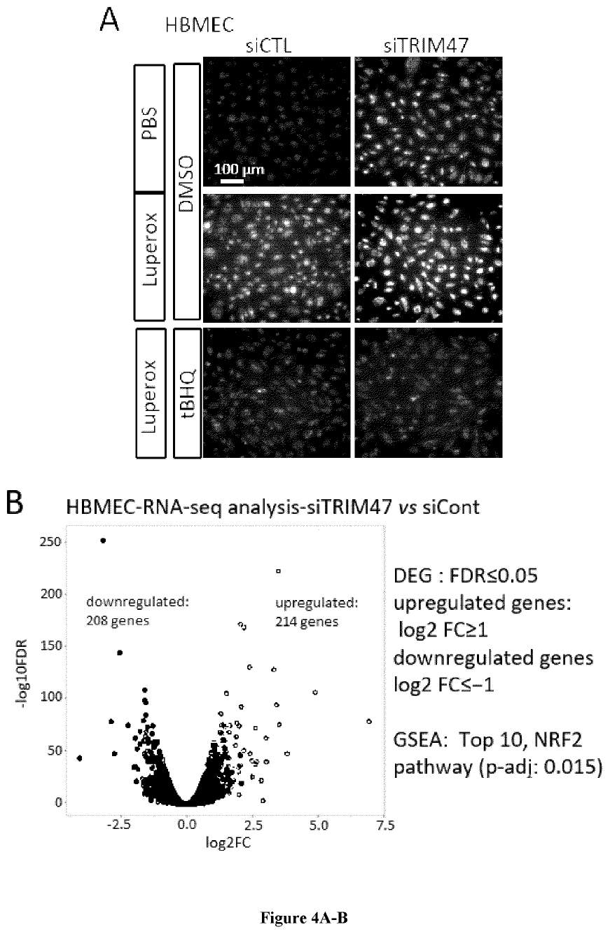

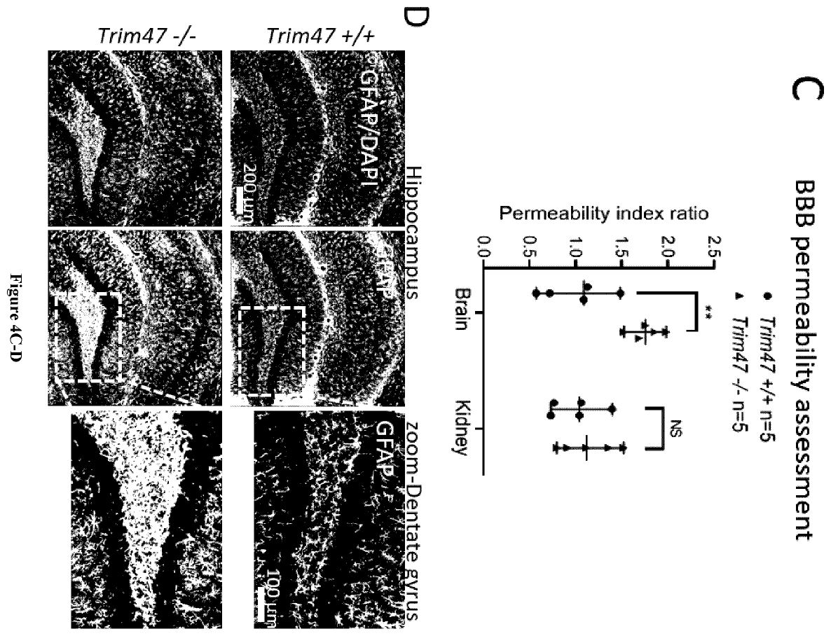

Discussion. These results suggest that endothelial TRIM47 is a key regulator of actin cytoskeleton organization through KEAP1/NRF2 signaling pathway and might be protective from oxidative stress in brain EC (FIGS. 1, 2 and 3).

### Example 2

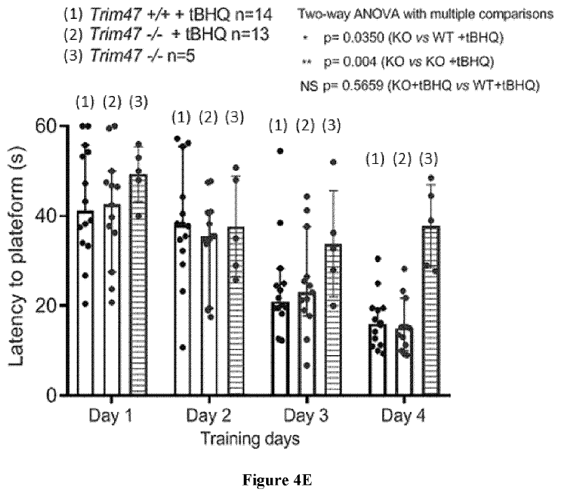

Human brain microvascular endothelial cells (HBMEC) were treated with control or TRIM47 siRNA and with/without Luperox (inducer of oxidative stress) and incubated with Cellrox Green dye (detection of oxidative stress). A pre-treatment with tert-butylhydroquinone (tBHQ) reversed induction of oxidative stress. FIG. 4A demonstrates that TRIM47 displays antioxidant properties.

RNA-sequencing was performed on HBMEC treated with control or TRIM47 siRNA. Volcano plot shows log 2 fold change (FC) vs −log 10FDR of differentially expressed genes (DEG) in response to TRIM47 knockdown. Gene set enrichment analysis revealed a significant (p<0.05) downregulation of genes involved in the NRF2 signalling pathway (FIG. 4B).

Assessment of blood brain barrier (BBB) permeability was done on Trim47 +/+ and Trim47 −/− adult mice by measuring extravasation of a fluorescent tracer (cadaverin, 0.98 kDa, IV injection) in mouse tissues. Trim47 −/− mice display increased blood vessels permeability specifically in brain (BBB dysfunction) as shown by a significant increase in permeability index ratio (normalization to fluorescence detected in serum) in brain cortex but not in kidney (FIG. 4C).

FIG. 4D is a representative image by immunofluorescence of GFAP expression (activated astrocytes) in brain sections from Trim47 +/+ and Trim47 −/− adult mice. Data show increased GFAP expression in hippocampus in Trim47 −/− mice.

qPCR screening of Nrf2 and its target genes performed on brain endothelial cells isolated from adult mice with/without a 1-month diet with tBHQ (1% W/W) showed a significant impairment of the Nrf2 pathway in Trim47 −/− mice which was prevented by tBHQ treatment (Table 1).

Water maze behavioural test performed on adult mice with/without a diet with tBHQ revealed that Nrf2 pathway re-activation is sufficient to prevent cognitive impairment observed in Trim47 −/−. Latency to reach platform is shown for the 4 training days (FIG. 4E).

These results suggest that TRIM47 is essential to brain physiology by promoting the NRF2 antioxidant protective system.

TABLE 1
Expression profile on Brain EC. Relative gene
expression fold change (normalized to Pecam1).

| | | without diet | | | +tBHQ diet | |
|---|---|---|---|---|---|---|
| | | Trim47 WT | Trim47 KO | | Trim47 WT | Trim47 KO |
| | Trim47 | 1.030 | ND | Trim47 | 1.066 | ND |
| BBB | Cdh5 | 1.031 | 0.749 | Cdh5 | 1.013 | 0.967 |
| integrity | Cldn5 | 0.994 | 0.735 * | Cldn5 | 1.071 | 0.877 |
| | Cldn11 | 1.032 | 0.721 * | Cldn11 | 1.016 | 1.020 |
| | Ocln | 1.011 | 0.737 ** | Ocln | 1.025 | 0.895 |
| Nrf2 | Nfe2l2 | 1.068 | 1.204 | Nfe2l2 | 1.007 | 1.040 |
| pathway | Keap1 | 1.041 | 1.040 | Keap1 | 1.014 | 0.730 |
| | Hmox1 | 1.013 | 0.318 ** | Hmox1 | 1.033 | 1.095 |
| | Nqo1 | 1.017 | 0.414 ** | Nqo1 | 1.033 | 1.084 |
| | Gclm | 1.034 | 0.396 ** | Gclm | 0.999 | 1.009 |
| | Me1 | 1.041 | 0.402 ** | Me1 | 1.033 | 1.148 |
| | Slc7a11 | 1.026 | 0.337 ** | Slc7a11 | 1.030 | 1.145 |
| | | mean of n = 6 | mean of n = 6 | | mean of n = 5 | mean of n = 5 |

### REFERENCES

Throughout this application, various references describe the state of the art to which this invention pertains. The disclosures of these references are hereby incorporated by reference into the present disclosure.

1. A method of treating a cerebral small vessel disease in a patient in need thereof comprising administering to the patient a therapeutically effective amount of a Nrf2 activator.

2. The method of claim 1 wherein the Nrf2 activator is a chalcone derivatives.

3. The method of claim 1 wherein the Nrf2 activator is selected from the group consisting of 2-trifluoromethyl-2-methoxychalcone, auranofin, ebselen, 1,2-naphthoquinone, cinnamic aldehyde, caffeic acid and its esters, curcumin, reservatrol, artesunate, tert-butylhydroquinone, (tBHQ), tert-butyl-p-quinone (tBQ), vitamin K1, vitamin K2, vitamin K3, a fumaric acid ester, 2-cyclopentenones, ethacrynic acid and its alkyl esters, bardoxolone methyl (methyl 2-cyano-3,12-dioxooleana-1,9(11)dien-28-oate) (CDDO-Me, RTA 402), ethyl 2-cyano-3,12-dioxooleana-1,9(11)dien-28-oate, 2-cyano-3,12-dioxooleana-1,9(11)dien-28-oic acid (CDDO), 1 [2-Cyano-3,12-dioxooleana-1,9(11)-dien-28-oyl]imidazole (CDDO-Im), (2-cyano-N-methyl-3,12-dioxooleana-1,9(11)-dien-28 amide (CDDO-methyl amide, CDDO-MA), isothiocyanate, 1,2-dithiole-3-thione, 3,5-di-tert-butyl-4-hydroxytoluene, 3-hydroxycoumarin, 4-hydroxynonenal, 4-oxononenal, malondialdehyde, (E)-2-hexenal, capsaicin, allicin, allylisothiocyanate, 6-methylthiohexyl isothiocyanate, 7-methylthioheptyl isothiocyanate, sulforaphane, 8-methylthiooctyl isothiocyanate, a corticosteroids, 8-iso prostaglandin A2, alkyl pyruvate, diethyl oxaloproprionate, dimethyl oxaloproprionate, 2-acetamidoacrylate, methyl-2-acetamidoacrylate, ethyl-2-acetamidoacrylate, hypoestoxide, parthenolide, eriodictyol, 4-hydroxy-2-nonenal, 4-oxo-2nonenal, geranial, zerumbone, aurone, isoliquiritigenin, xanthohumol, [10]-shogaol, eugenol, 1-acetoxychavicol acetate, allyl isothiocyanate, benzyl isothiocyanate, phenethyl isothiocyanate, 4-(methylthio)-3-butenyl isothiocyanate, 6-methylsulfinylhexyl isothiocyanate, ferulic acid and its esters, sofalcone, 4-methyl daphnetin, imperatorin, auraptene, poncimarin, a bis[2-hydroxybenzylidene]acetone, alicylcurcuminoid, 4-bromo flavone, β-naphthoflavone, sappanone A, an aurone or an indole derivative of an aurone, perillaldehyde, quercetin, fisetin, koparin, genistein, tanshinone, a hydroxy acid (HA), a beta hydroxy acid (BHA), butylated hydroxytoluene (BHT), PMX-290, AL-1, avicin D, gedunin, fisetin, andrographolide, tricyclic bis(cyanoenone) (TBE-31), [(±)-(4bS,8aR,10aS)-10a-ethynyl-4-b,8,8-trimethyl-3,7-dioxo-3,4-b,7,8,8a,9,10,10a-octahydrophenanthrene-2,6-dicarbonitrile], MCE-1, MCE5, and TP-225.

4. The method of claim 1 wherein the Nrf2 activator is dimethyl fumarate.

5. The method of claim 1, wherein the Nrf2 activator is diroximel fumarate.

6. The method of claim 3, wherein the vitamin K3 is menadione.

7. The method of claim 3, wherein the fumaric acid ester is a fumaric acid monoester or a fumaric acid diester.

8. The method of claim 7, wherein the fumaric acid monoester is a monoalkyl hydrogen fumarate and the fumaric acid diester is a dialkyl fumarate or a metabolite of dialkyl fumarate.

9. The method of claim 8, wherein the monoalkyl hydrogen fumarate is monomethyl hydrogen fumarate.

10. The method of claim 8, wherein the dialkyl fumarate is diethyl fumarate.

11. The method of claim 8, wherein the metabolite of dialkyl fumarate is diroximel fumarate.

12. The method of claim 8, wherein the indole derivative of an aurone is benzylidene-indolin-2-one.
